(19) United States
(12) Patent Application Publication
(10) Pub. No.: US 2024/0342010 A1
(43) Pub. Date: Oct. 17, 2024

(54) FOOT PROTECTION SYSTEM

(71) Applicant: LaTasha Tillman, Walled Lake, MI (US)

(72) Inventor: LaTasha Tillman, Walled Lake, MI (US)

(21) Appl. No.: 18/135,176

(22) Filed: Apr. 16, 2023

(51) Int. Cl. A61F 13/06; A61F 13/00

(52) U.S. Cl. CPC A61F 13/064; A61F 2013/00404

(57) ABSTRACT

A foot protection system, including at least one toe pad worn on at least one toe of a foot to prevent friction during movement of the at least one toe pad on a ground surface, and at least one ball pad worn on a ball of the foot to prevent friction during movement of the at least one bail pad on the ground surface.

### BACKGROUND

### 1. Field

The present general inventive concept relates generally to foot padding, and particularly, to a foot protection system.

### 2. Description of the Related Art

Dancing is a type of performing art that uses a sequence of movements using different parts of a body. Moreover, dancing often uses feet during movements that puts a lot of pressure on the feet.

Many dancers prefer dancing while barefoot to improve aesthetic and dexterity on a ground surface. However, dancing barefoot in over a long period of time can cause blisters which is not only physically painful, but can also hinder a dancer's performance and overall comfort.

Therefore, there is a need for a foot protection system that can be worn on the feet to protect the feet during dancing.

### SUMMARY

The present general inventive concept provides a foot protection system.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a foot protection system, including at least one toe pad worn on at least one toe of a foot to prevent friction during movement of the at least one toe pad on a ground surface, and at least one ball pad worn on a ball of the foot to prevent friction during movement of the at least one ball pad on the ground surface.

The at least one toe pad and the at least one ball pad, each may include a ground contacting surface to reduce a friction level during movement on the ground surface, and a fastening surface disposed on at least a portion of the ground contacting surface to connect the ground contacting surface to the foot.

The ground contacting surface may be constructed of suede.

The ground contacting surface may have a color of skin.

The at least one toe pad may have a size and a shape corresponding to the at least one toe of the foot and the at least one ball pad has a size and a shape corresponding to the ball of the foot.

The foot protection system may further include a lubricant chamber disposed within at least a portion of the at least one toe pad and the at least one ball pad to dispense at least one lubricant in response to movement of the at least one foot pad or the at least one ball pad on the ground surface exceeding a predetermined friction level.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front view of a foot protection system as disposed on feet, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Foot Protection System 100
Toe Pads 110
Ground Contacting Surface 111
Fastening Surface 112
Ball Pads 120
Ground Contacting Surface 121
Fastening Surface 122
Lubricant Chamber 130

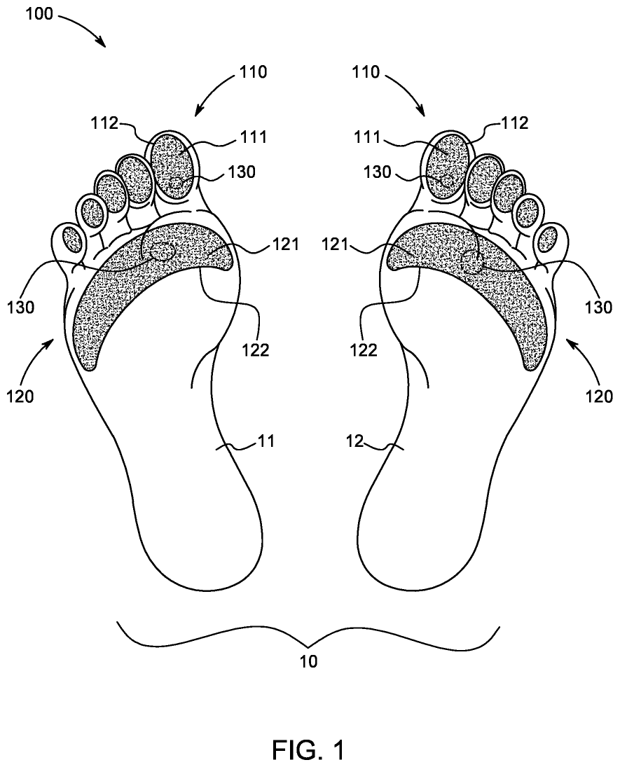

FIG. 1 illustrates a front view of a foot protection system 100 as disposed on feet 10, according to an exemplary embodiment of the present general inventive concept.

The foot protection system 100 may be constructed from at least one of cloth, wool, plastic, and rubber, etc., but is not limited thereto.

The foot protection system 100 may include a plurality of toe pads 110, a plurality of ball pads 120, and a lubricant chamber 130, but is not limited thereto.

Each of the plurality of toe pads 110 may include a ground contacting surface 111 and a fastening surface 112, but is not limited thereto.

The ground contacting surface 111 may be constructed of suede and foam, but is not limited thereto. Also, the ground contacting surface 111 may have a color of skin and/or flesh of a user based on a preference of the user to obscure visibility from at least one third person during dancing. The ground contacting surface 111 may absorb an impact (e.g., striking, contact, falling) thereon, such as from a ground surface. Moreover, the ground contacting surface 111 may prevent damage and/or injury to a first foot 11 and/or a second foot 12 of the user. More specifically, the ground contacting surface 111 may facilitate movement (e.g., sliding, slipping) on the ground surface to prevent friction and/or floor burn by reducing a friction level during movement on the ground surface.

The fastening surface 112 may include an adhesive (e.g., tape, glue), a cord, a string, a wire, rope, a clip, a clamp, a clasp, a snap, a magnet, and/or any combination thereof, but is not limited thereto.

The fastening surface 112 may be disposed on at least a portion of the ground contacting surface 111. The fastening surface 112 may removably connect (e.g., adhere, affix, stick, wrap around, tie around, clamp) to at least a portion of the first foot 11 and/or the second foot 12 of the user, such that the ground contacting surface 121 may be worn by the user. For example, the fastening surface 112 may removably connect to at least one toe of the user. In other words, the ground contacting surface 111 and/or the fastening surface 112 may have a size and/or a shape corresponding to a bottom surface of the at least one toe of the user. Also, the fastening surface 112 may prevent the ground contacting surface 111 from being removed from the first foot 11 and/or the second foot 12.

The fastening surface 112 may be designed to be reusable for repeated use of the ground contacting surface 111.

Each of the plurality of ball pads 120 may include a ground contacting surface 121 and a fastening surface 122, but is not limited thereto.

The ground contacting surface 121 may be constructed of suede and foam, but is not limited thereto. Also, the ground contacting surface 121 may have a color of skin and/or flesh of the user based on a preference of the user to obscure visibility from at least one third person during dancing. The ground contacting surface 121 may absorb an impact (e.g., striking, contact, falling) thereon, such as from a ground surface. Moreover, the ground contacting surface 121 may prevent damage and/or injury to the first foot 11 and/or the second foot 12 of the user. More specifically, the ground contacting surface 121 may facilitate movement (e.g., sliding, slipping) on the ground surface to prevent friction and/or floor burn by reducing a friction level during movement on the ground surface.

The fastening surface 122 may include an adhesive (e.g., tape, glue), a cord, a string, a wire, rope, a clip, a clamp, a clasp, a snap, a magnet, and/or any combination thereof, but is not limited thereto.

The fastening surface 122 may be disposed on at least a portion of the ground contacting surface 121. The fastening surface 122 may removably connect (e.g., adhere, affix, stick, wrap around, tie around, clamp) to at least a portion of the first foot 11 and/or the second foot 12 of the user, such that the ground contacting surface 121 may be worn by the user. For example, the fastening surface 122 may removably connect to a ball of the first foot 11 and/or a ball of the second foot 12 of the user. In other words, the ground contacting surface 121 and/or the fastening surface 112 may have a size and/or a shape corresponding to the ball of the first foot 11 and/or the ball of the second foot 12. Also, the fastening surface 122 may prevent the ground contacting surface 121 from being removed from the first foot 11 and/or the second foot 12.

The fastening surface 122 may be designed to be reusable for repeated use of the ground contacting surface 121.

The lubricant chamber 130 may be disposed within at least a portion of each of the plurality of toe pads 110 between the ground contacting surface 111 and/or the fastening surface 112. Also, the lubricant chamber 130 may be disposed within at least a portion of each of the plurality of ball pads 120 between the ground contacting surface 121 and/or the fastening surface 122. The lubricant chamber 130 may store at least one lubricant therein, such as oil, deionized water, polyoxyethylene, sodium-carbomer, phenoxyethanol, and/or ethylhexylglycerin.

The lubricant chamber 130 may be constructed to rupture in response to movement of the ground contacting surface 111 and/or the ground contacting surface 121 on the ground surface exceeding a predetermined stress level and/or a friction level of the ground contacting surface 111 and/or the ground contacting surface 121, respectively. Additionally, the lubricant chamber 130 may dispense the at least one lubricant therefrom in response to rupturing to facilitate movement on the ground surface, such as during dancing. Thus, the lubricant chamber 130 may prevent injury to the feet 10 of the user during dancing.

Therefore, the foot protection system 100 may protect the feet 10 of the user during dancing. Also, the foot protection system 100 may allow the user to dance barefoot and increase aesthetic appeal during dancing.

The present general inventive concept may include a foot protection system 100, including at least one toe pad 110 worn on at least one toe of a foot 11/12 to prevent friction during movement of the at least one toe pad 110 on a ground surface, and at least one ball pad 120 worn on a ball of the foot 11/12 to prevent friction during movement of the at least one ball pad 120 on the ground surface.

The at least one toe pad 110 and the at least one ball pad 120, each may include a ground contacting surface 111/121 to reduce a friction level during movement on the ground surface, and a fastening surface 112/122 disposed on at least a portion of the ground contacting surface 111/121 to connect the ground contacting surface to the foot 11/12.

The ground contacting surface 111/121 may be constructed of suede.

The ground contacting surface 111/121 may have a color of skin.

The at least one toe pad 110 may have a size and a shape corresponding to the at least one toe of the foot 11/12 and the at least one ball pad 120 has a size and a shape corresponding to the ball of the foot 11/12.

The foot protection system 100 may further include a lubricant chamber 130 disposed within at least a portion of the at least one toe pad 110 and the at least one ball pad 120 to dispense at least one lubricant in response to movement of the at least one foot pad 110 or the at least one ball pad 120 on the ground surface exceeding a predetermined friction level.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A foot protection system, comprising:
at least one toe pad worn on at least one toe of a foot to prevent friction during movement of the at least one toe pad on a ground surface; and
at least one ball pad worn on a ball of the foot to prevent friction during movement of the at least one ball pad on the ground surface.

2. The foot protection system of claim 1, wherein the at least one toe pad and the at least one ball pad, each comprise:
a ground contacting surface to reduce a friction level during movement on the ground surface; and
a fastening surface disposed on at least a portion of the ground contacting surface to connect the ground contacting surface to the foot.

3. The foot protection system of claim 2, wherein the ground contacting surface is constructed of suede.

4. The foot protection system of claim 2, wherein the ground contacting surface has a color of skin.

5. The foot protection system of claim 1, wherein the at least one toe pad has a size and a shape corresponding to the at least one toe of the foot and the at least one ball pad has a size and a shape corresponding to the ball of the foot.

6. The foot protection system of claim 1, further comprising:
a lubricant chamber disposed within at least a portion of the at least one toe pad and the at least one ball pad to dispense at least one lubricant in response to movement of the at least one foot pad or the at least one ball pad on the ground surface exceeding a predetermined friction level.
